(19) United States
(12) Patent Application Publication
(10) Pub. No.: US 2007/0055218 A1
(43) Pub. Date: Mar. 8, 2007

(54) Tubular assembly

(75) Inventor: Pavel Menn, Marblehead, MA (US)

Correspondence Address: GREENBLUM & BERNSTEIN, P.L.C., 1950 ROLAND CLARKE PLACE, RESTON, VA, 20191, US

(73) Assignee: MICROLINE PENTAX Inc., Beverly, MA (US)

(21) Appl. No.: 11/202,162

(22) Filed: Aug. 12, 2005

(51) Int. Cl. A61B 17/00

(52) U.S. Cl. 606/1

(57) ABSTRACT

A tubular assembly such that the assembly remains attached to a tubular structure using a collar and without need for extra parts and also such that a fluid resistant seal is formed between the assembly and the structure. The assembly has a flare on one end and a detent a fixed distance from the flare. The collar is rotatable and twistably or threadably attachable to the tubular structure.

### BACKGROUND OF THE INVENTION

1. Field of the Invention

The present invention is related to a tube assembly which allows for connection to a separate tubular structure with a collar at the connecting point of the two parts, and more particularly to such a tubular assembly used in medical instruments like one for stapling a patient's body.

2. Background and Material Information

It is important for medical instruments with more than one part to be assembled such that they are well sealed, to prevent any possible leakage of fluid from the body into the instrument, and to be well put together, to negate the possibility of the instrument disassembling at an inopportune moment when the user does not want it to. If the instrument is disposable, than it can simply be assembled such that the parts are immovably attached to each other and then thrown away whole after use. If the instrument is re-usable, the problem becomes more difficult. The related art generally involves the use of an o-ring or gasket to prevent fluid leakage and of an extra fastening device, beyond the parts of the instrument itself, such as a lock screw or the like to prevent accidental disassembly. With these extra parts comes extra cost and extra assembly time. The related art can also involve a collet to prevent a collar or surrounding part from accidentally coming off the instrument. The collet is attached via welding or a fastening substance to one of the parts of the instrument and thus limits the movement of the collar along that part of the instrument in one direction. However, use of the collet necessitates extra parts as well as extra assembly time and processes and could potentially lead to problems as the collet is capable of falling off the instrument if the seal between the two breaks.

### SUMMARY OF THE INVENTION

The present application provides a tube assembly for attachment to and removal from a tubular structure, where the tubular structure may be part of a medical instrument configured operate a medical implement and the tube assembly may be configured to accept the medical implement therein. The tube assembly may have a flare portion at a first end of the tube assembly; a detent formed in a wall of the tube assembly, such that the region in which the detent is formed is of generally uniform thickness, at a predetermined distance from the flare portion; and a rotatable collar positioned about the tube assembly in order to twistably attach to the tubular structure such that the flare portion abuts the tubular structure to form a fluid-resistant seal between the tube assembly and the tubular structure and the movement of the collar along an axial length of the tube assembly is limited in a first direction by the flare portion and in a second direction by the detent.

The aforementioned detent may continuously or discontinuously encircle the tube assembly. Also, the detent may be rounded. Further, the collar may be configured to threadably attach to the tubular structure.

Another feature further provides a barrel assembly which may include a hollow tube having a first end configured to engage a medical instrument; a flare portion at the first end of the hollow tube; a rounded detent, which may continuously or discontinuously encircle the hollow tube, formed in a wall of the hollow tube, where the region of the wall in which the detent is formed if of generally uniform thickness, at a predetermined distance from the flare portion; and a rotatable collar positioned about the first end of the hollow tube that twistably attaches to the medical instrument such that the flare portion abuts the medical instrument to form a fluid-resistant seal between the hollow tube and the medical instrument, wherein movement of the collar along an axial length of the hollow tube is limited in a first direction by the flare portion and in a second direction by the detent. Additionally, the hollow tube may be configured to accept a medical implement and the collar may be configured to threadably attach to the medical instrument.

The present application also provides a medical instrument that may include a housing, a barrel configured to attach to and be removed from the housing, a flare portion at a first end of the barrel, a rounded detent which may continuously or discontinuously encircle the barrel formed in a wall of the barrel such that the thickness of the wall in the region in which the detent is formed is generally uniform at a predetermined distance from the flare portion, and a rotatable collar configured to twistably or threadably attach to the housing such that the flare portion abuts the housing to form a fluid-resistant seal between the barrel and the housing, wherein movement of the collar along an axial length of the barrel is limited in a first direction by the flare portion and in a second direction by the detent. Further the medical instrument may include a medical implement configured to effect an operation on a body, wherein the barrel is configured to accept said medical implement therein.

The detent used in the present invention has several advantages over the prior art. First, the detent is formed in the instrument or device itself and thus eliminates the need for extra parts. Second, the formation of the detent simplifies the manufacture process over the process needed to attach extra parts such as locking screws or a collet or both. Third, as the detent is formed in the instrument or device itself, it cannot disassemble and, therefore, will always serve its function.

Other exemplary embodiments and advantages of the present invention may be ascertained by reviewing the present disclosure and the accompanying drawing, and the above description should not be considered to limit the scope of the present application.

### BRIEF DESCRIPTION OF THE DRAWINGS

The present invention is further described in the detailed description which follows, in reference to the noted plurality of drawings, by way of non-limiting examples of preferred embodiments of the present invention, in which like numerals represent like elements throughout the drawing, and wherein:

FIG. 1 is a side cross-sectional view of a tube assembly according to the embodiment of the present invention.

### DETAILED DESCRIPTION OF THE PREFERRED EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice.

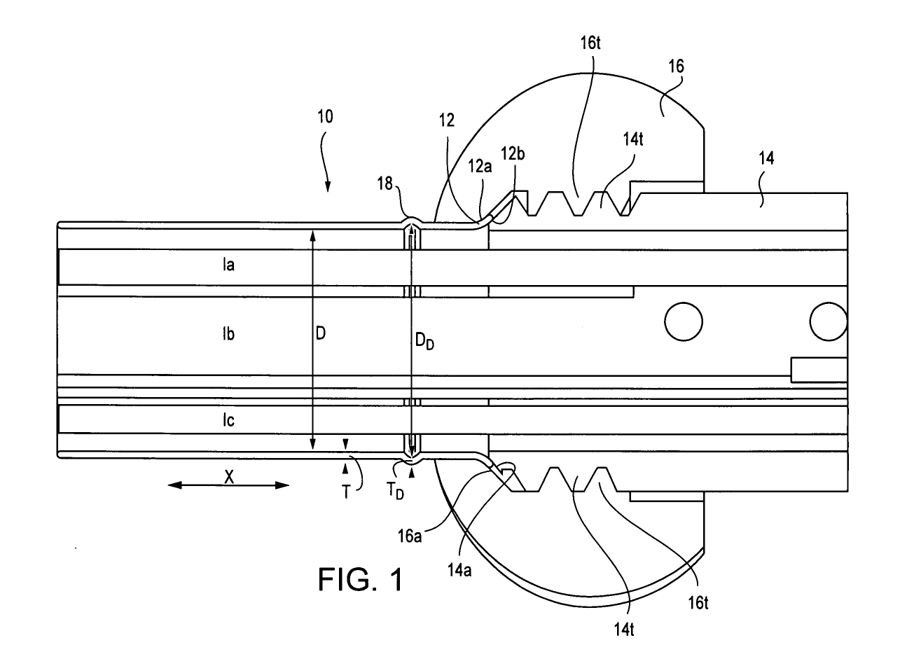

Referring to the drawing wherein like numerals represent like elements, FIG. 1 shows a tube assembly 10 that can be removably connected, at a proximal end thereof, to a separate tubular structure 14, via a collar 16 at the connection point of the parts. The tube assembly 10 is particularly useful in a medical instrument but can be used in any device or instrument where it is necessary to connect two tubular pieces. Also, the space inside of tubular assembly 10 is capable of receiving a device or instrument, Iₐ, I_{b}, I_{c}, for example a medical implement such as a clip applier cartridge. The assembly 10 is described as tubular but it is understood by those skilled in the art that it can be any shape that will connect with the structure 14 including, but not limited to e.g., oval, rectangular, and triangular. The tubular structure 14 is described as tubular shaped but it is understood by those skilled in the art that it can be any shape that will connect with the tubular assembly 10 including, but not limited to e.g., oval, rectangular, and triangular.

As shown in FIG. 1, the tube assembly 10 may have a flare portion 12 and a detent 18. The detent 18 is unitarily created in the wall of the tube assembly 10, to avoid the welding of a separate collet. The thickness of the wall of the tube assembly 10 at the detented portion, T_{D}, may be approximately the same as the thickness of the wall of the tube assembly 10 elsewhere, T. However, it is not required that the thickness elsewhere, T, be the same throughout the entire tube assembly 10. Also, the inner diameter, D_{D}, of the portion of the tube assembly 10 in which the detent 18 occurs may be larger than the inner diameter, D, of the tube assembly elsewhere. However, it is not required that the inner diameter elsewhere, D, be the same throughout the entire tube assembly 10. IN other words, the wall thickness T and/or the inner diameter D may be varied, depending on the configuration of the instrument. The detent may be continuous around the circumference of the tube assembly 10 or it may be interrupted or discontinuous; for example it may be a series of raised circular protrusions or a series or linear portions. Also, the detent, which can be formed by rolling pressing or other such manufacturing processes, is shown as being semi-circular in shape but it is understood by one in the art that it could be any shape including but not limited to square, triangular, or trapezoidal.

The tubular structure 14 may be connected to the tube assembly 10 in order to form a single device or instrument where the collar 16 may be at the place of juncture. The collar 16 may be tapped so that the collar 16 can twistably or threadably attach to the tubular structure 14 such that the internal threads 16t of the collar may fit with the external threads 14t of the tubular structure. However, the collar 16 and tubular structure 14 may alternatively be made to attach in a non-threadable manner including but not limited to attaching via bayonet, snap-fit, or press-fit.

The inner surface of the flare portion 12b may abut the outer edge portion of the tubular structure 14a thus forming a generally fluid-resistant seal. The outer surface of the flare portion 12a may abut the inner surface 16a of the threaded portion of the collar. This placement of the flare portion of the tube assembly 12 and the tubular structure 14 may allow for the collar 16 to maintain connection between the tube assembly 10 and tubular structure 14. In the attached position, shown in FIG. 1, movement of the collar 16 along longitudinal axis X in a proximal (or first) direction (i.e., to the right in FIG. 1) is restricted by the flare portion 12, to prevent overtightening and/or to prevent disengagement of the collar 16 from the tube assembly 10.

Thus, the tube assembly 10 shown in FIG. 1 may remain attached to the tubular assembly 14 with only the collar 16 and no other fasteners. Further, the tube assembly and tubular structure form a fluid-resistant seal without the use of sealant such as o-rings. However, it is also possible, and may in certain circumstances be helpful, to use extra fasteners or sealants in conjunction with the embodiment shown.

When the tube assembly 10 is detached from the tubular structure 14 (e.g., for sterilization), movement of the collar along the longitudinal axis X in the proximal direction is restricted by the flare portion 12, and movement of the collar in a distal (or second) direction (i.e., to the left in FIG. 1) is restricted by the detent 18, thereby preventing the collar from sliding off of the tube assembly in either direction.

It is noted that the foregoing examples have been provided merely for the purpose of explanation and are in no way to be construed as limiting of the present invention. While the present invention has been described with reference to a preferred embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed:

1. A tube assembly for attachment to and removal from a tubular structure, the tube assembly comprising:
a flare portion at a first end of the tube assembly;
a detent formed in a wall of the tube assembly at a predetermined distance from said flare portion;
a rotatable collar positioned about the tube assembly and being twistably attachable to the tubular structure such that said flare portion abuts the tubular structure to form a fluid-resistant seal between the tube assembly and the tubular structure, wherein movement of said collar along an axial length of the tube assembly is limited in a first direction by said flare portion and in a second direction by said detent.

2. The tube assembly according to claim 1, wherein said detent continuously encircles the tube assembly.

3. The tube assembly according to claim 1, wherein said detent discontinuously encircles the tube assembly.

4. The tube assembly according to claim 1, wherein:
the tubular structure is part of a medical instrument; and
the tube assembly is configured to accept a medical implement therein.

5. The tube assembly according to claim 1, wherein said collar is threadably attachable to the tubular structure.

6. The tube assembly according to claim 1, wherein said detent is rounded.

7. The tube assembly according to claim 1 wherein at least a region of the wall in which said detent is formed is of a generally uniform thickness.

8. A barrel assembly comprising:
a hollow tube having a first end configured to engage a medical instrument;
a flare portion at said first end of said hollow tube;
a detent formed in a wall of said hollow tube at a predetermined distance from said flare portion;
a rotatable collar positioned about said first end of said hollow tube and being twistably attachable to the medical instrument such that said flare portion abuts the medical instrument to form a fluid-resistant seal between said hollow tube and said medical instrument, wherein movement of said collar along an axial length of said hollow tube is limited in a first direction by said flare portion and in a second direction by said detent.

9. The barrel assembly according to claim 8, wherein said detent continuously encircles said hollow tube.

10. The barrel assembly according to claim 8, wherein said detent discontinuously encircles said hollow tube.

11. The barrel assembly according to claim 8, wherein said hollow tube is configured to accept a medical implement therein.

12. The barrel assembly according to claim 8, wherein said collar is threadably attachable to the medical instrument.

13. The barrel assembly according to claim 8, wherein said detent is rounded.

14. The barrel assembly according to claim 8, wherein a region of the wall in which the detent is formed is of a generally uniform thickness.

15. A medical instrument comprising:
a housing;
a barrel attachable to and removable from said housing;
a flare portion at a first end of said barrel;
a detent formed in a wall of said barrel at a predetermined distance from said flare portion;
a rotatable collar twistably attachable to said housing such that said flare portion abuts said housing to form a fluid-resistant seal between said barrel and said housing, wherein movement of said collar along an axial length of said barrel is limited in a first direction by said flare portion and in a second direction by said detent.

16. The instrument according to claim 15, wherein said detent continuously encircles said barrel.

17. The instrument according to claim 15, wherein said detent discontinuously encircles said barrel.

18. The instrument according to claim 15, further comprising a medical implement configured to effect an operation on a body, wherein said barrel is configured to accept said medical implement therein.

19. The instrument according to claim 15, wherein said collar is threadably attachable to said housing.

20. The instrument according to claim 15, wherein said detent is rounded.

21. The instrument according to claim 15, wherein a region of the wall in which said detent is formed is of a generally uniform thickness.
